(19) United States
(12) Patent Application Publication
(10) Pub. No.: US 2022/0275317 A1
(43) Pub. Date: Sep. 1, 2022

(54) A BIOPROCESS SYSTEM

(71) Applicant: Cytiva Sweden AB, Uppsala (SE)

(72) Inventors: Klas Marteleur, Uppsala (SE); Andreas Marcstrom, Uppsala (SE); Mats Olsson, Uppsala (SE); Klaus Gebauer, Uppsala (SE)

(21) Appl. No.: 17/763,135

(22) PCT Filed: Sep. 30, 2020
(86) PCT No.: PCT/EP2020/077365

§ 371 Date: Mar. 23, 2022

(30) Foreign Application Priority Data
Oct. 1, 2019 (GB) 1914176.1

(51) Int. Cl. C12M 1/12; C12M 1/00; C12M 1/36; B33Y 80/00

(52) U.S. Cl. CPC C12M 23/06; C12M 23/40; C12M 29/20; C12M 41/48; B33Y 80/00

(57) ABSTRACT

Disclosed is a bioprocess system (1) comprising a flow path (3; 3a, 3) comprising: a plurality of inlets (7); at least one bio-processing device inlet connection (9a) and at least one bioprocessing device outlet connection (9b); a plurality of outlets (11); and flow path parts (5a′, 5b′, 5c′, 5d′, 5e′) of at least some active components (5a, 5b, 5c, 5d, 5e) comprised in the bioprocess system; and active parts (5a″, 5b″, 5c″, 5d″, 5e″) of said active components (5a, 5b, 5c, 5d, 5e). The flow path (3; 3a, 3b) of the bioprocess system is positioned centrally within the bioprocess system (1) and in a vertical orientation, whereby the active parts (5a″, 5b″, 5c″, 5d″, 5e″) are positioned around the flow path (3; 3a, 3b) being accessible from outside the bioprocess system (1) and each in connection with a corresponding flow path part (5a′, 5b′, 5c′, 5d′, 5e′) of an active component (5a, 5b, 5c, 5d, 5e).

### TECHNICAL FIELD

The present invention relates to a bioprocess system.

### BACKGROUND

A bioprocess system comprises usually a number of inlets, outlets, pumps, valves, sensors and flow connections to a connected bioprocessing device, such as for example a separation unit, such as for example a chromatography column or a filtering unit. Pipes for fluid connections in the system are usually welded to the different components. The systems are often large and need to be placed in a clean room. Furthermore they need to be possible to sanitize and service. Connection pipes and welds between pipes and components may decrease sanitation effectiveness and increase hold up volumes in the system.

### SUMMARY

An object of the present invention is to provide an improved bioprocess system.

A further object of the present invention is to provide an effective bioprocess system with a small footprint.

This is addressed, for example, by a bioprocess system according to claim 1.

According to one aspect of the invention a bioprocess system is provided comprising a flow path comprising: a plurality of inlets; at least one bioprocessing device inlet connection and at least one bioprocessing device outlet connection; a plurality of outlets; and flow path parts of at least some active components comprised in the bioprocess system; and active parts of said active components, wherein the flow path of the bioprocess system is positioned centrally within the bioprocess system and in a vertical orientation, whereby the active parts are positioned around the flow path being accessible from outside the bioprocess system and each in connection with a corresponding flow path part of an active component.

Hereby, by providing the flow path centrally within the bioprocess system and in a vertical direction both the footprint of the system can be kept small and purging of air in the system will be improved. Furthermore, by providing the flow path centrally and active parts of active components around the flow part, the active parts are easily accessed from outside the bioprocess system. Hereby service is facilitated.

In one embodiment of the invention the flow path is 3D printed in a number of sections or in one section.

In one embodiment of the invention the flow path is at least to some degree self-supporting.

In one embodiment of the invention the flow path is 3D printed with a thickness which is adapted such that the flow path is at least to some degree self-supporting, which thickness may vary for different parts of the flow path and/or wherein the flow path is 3D printed together with additional external support structures.

In one embodiment of the invention said active components comprise a number of valves and possibly also one or more pumps and one or more sensing components.

In one embodiment of the invention said flow path is 3D printed from a corrosion resistant metal.

In one embodiment of the invention the bioprocess system further comprises a support structure which is a central spine provided inside the flow path or a frame which is partly surrounding the flow path, and which comprises user interface and tube connections.

### BRIEF DESCRIPTION OF THE DRAWINGS

FIG. 1a is a view from a first side of a bioprocess system according to one embodiment of the invention.

FIG. 1b is a view from a second side, which is opposite the first side of the same bioprocess system as shown in FIG. 1a.

FIG. 1c is a perspective view of the first side of the same bioprocess system as shown in FIG. 1a.

FIG. 1d is a perspective view of the second side of the same bioprocess system as shown in FIG. 1a.

FIGS. 2a and 2b are two different perspective views of a first side of a bioprocess system according to one embodiment of the invention.

FIGS. 2c and 2d are two different perspective views of a second side, which is opposite the first side of the same bioprocess system as shown in FIGS. 2a and 2b.

FIGS. 3a and 3b are two different perspective views of a bioprocess system according to one embodiment of the invention.

FIGS. 4a-4d show in perspective two separate parts of the fluid path of the bioprocess system as shown in FIGS. 1a-1d.

FIG. 5 is a perspective view of a bioprocess system according to another embodiment of the invention.

### DETAILED DESCRIPTION OF EMBODIMENTS

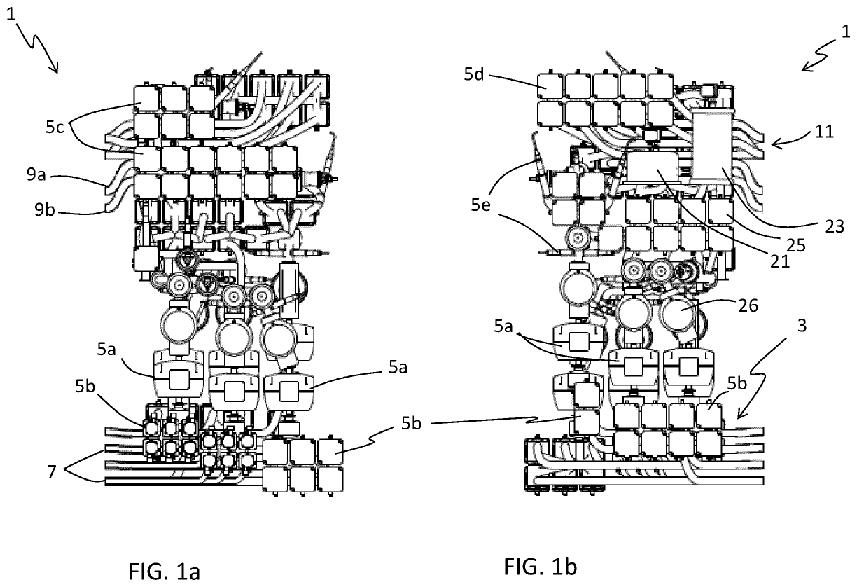

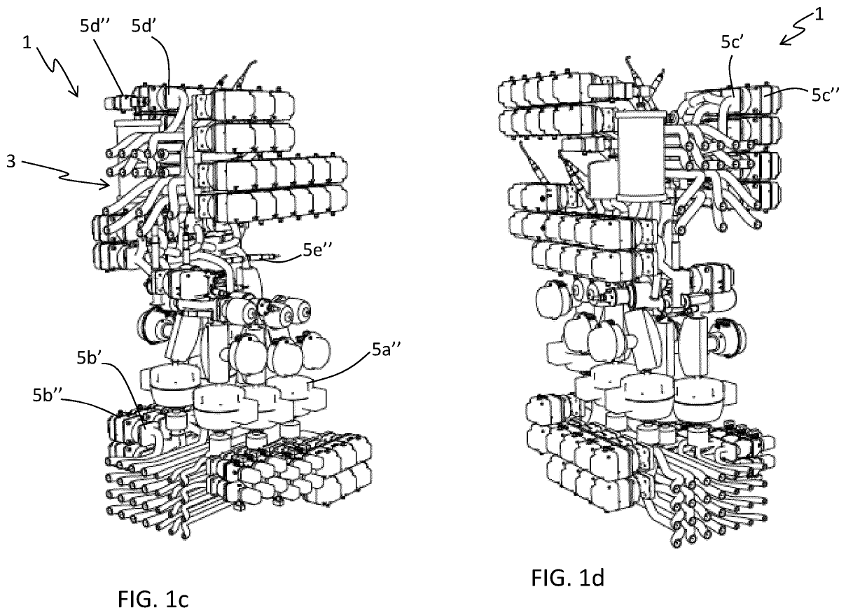

A bioprocess system 1 according to one embodiment of the invention is shown in FIGS. 1a-1d. FIG. 1a is a view from a first side, FIG. 1b is a view from a second side, which is opposite the first side and FIGS. 1c and 1d are perspective views. The bioprocess system 1 comprises a flow path 3 and a number of active components 5a, 5b, 5c, 5d, 5e. Said active components comprise a number of valves 5a, 5c, 5d and possibly also at least one pump 5a and a number of sensing components 5e. The active components each comprises a flow path part 5a′, 5b′, 5c′, 5d′, 5e′ and an active part 5a″, 5b″, 5c″, 5d″, 5e″. The active part can be a drive 5a″ of a pump, an actuating part 5b″, 5c″, 5d″ of a valve or a sensing part 5e″ of a sensor. The bioprocess system 1 can also comprise other components which will be described below.

The flow path 3 comprises a plurality of inlets 7, at least one bioprocessing device inlet connection 9a, at least one bioprocessing device outlet connection 9b and a plurality of outlets 11. The flow path 3 comprises furthermore the flow path parts 5a′, 5b′, 5c′, 5d′, 5e′ of the active components 5a, 5b, 5c, 5d, 5e.

In this embodiment of the invention one inlet valve 5b is provided to each inlet 7 and one outlet valve 5d is provided to each outlet 11. Furthermore a bioprocessing device valve 5c is provided to each bioprocessing device inlet and outlet connection 9a, 9b.

Other components which are provided in the bioprocess system 1 according to this embodiment are a filter 21 and a bubble trap 23. Valves 25 are also provided for controlling said filter 21 and said bubble trap 23. However these components and valves are not necessary in all embodiments. Furthermore some flow meters 26 and sensors 27 are provided in this embodiment which may not be always necessary and/or may be positioned differently in the system. A bioprocessing device, such as for example a separation or fluid treatment device, such as for example a chromatography column, a filter or a virus inactivation chamber (reactor) can be connected to the bioprocessing device inlet and outlet connections 9a, 9b and be a part of the bioprocess system 1 according to the invention or be a separate part.

According to the invention the flow path 3 of the bioprocess system 1 is positioned substantially centrally within the bioprocess system 1 and in a substantially vertical orientation. Furthermore the active parts 5a″, 5b″, 5c″, 5d″, 5e″ are positioned substantially around the flow path 3 being accessible from outside the bioprocess system and each in connection with a corresponding flow path part 5a′, 5b′, 5c′, 5d′, 5e′ of an active component 5a, 5b, 5c, 5d, 5e. Thanks to the central and vertical positioning of the flow path 3 the active components 5a, 5b, 5c, 5d, 5e can be easily accessed for service from outside the bioprocess system 1. Furthermore the inlets 7 of the flow path 3 are provided at a bottom part of the bioprocess system 1 and the outlets 11 are provided at a top part of the bioprocess system. Hereby, and thanks to the vertical orientation of the flow path 3 an upward flow can be kept in the bioprocess system 1 which will improve purging of air from the system.

The flow path 3 is according to the invention at least partly 3D printed. Either the whole flow path 3 can be 3D printed in one part or it could be printed in a number of sections. Materials for 3D printing the flow path 3 can be corrosion resistant metals, for example stainless steel or polymers with high chemical resistance such as for example polypropylene or PEEK. Thanks to the 3D printing of the flow path 3 the flow path can be made much more compact than in prior art systems. Welds and TC connections between different components can be avoided which will both save space and improve sanitizing. Furthermore connecting tubes can be printed with any degree of bending which will allow components to be placed closer to each other. Furthermore 3D printed components can be made smaller and more compact than with other production methods. Hereby a 3D printed flow path 3 according to the invention can be made much more compact than flow paths in any prior art system. Components will be provided closer giving a higher density of components in the bioprocess system. Furthermore, by providing the flow path 3 central and vertical in the bioprocess system 1 the footprint can be kept small.

For example, in various embodiments, the flow path 3 of the bioprocess system effectively replaces many conventional parts (connectors, clamps, seals, threads, welds, etc.) using fewer individual 3D printed components. 3D printing of parts is thus useful to enable a reduction in the number of connectors used, and also provides the further advantages of providing a compact system that is both easier to assemble and maintain, and which also reduces the chances of contaminants inadvertently entering the bioprocess system during assembly. The replacement ratio of conventional parts to the number of connectors needed to provide the flow path 3 is thus low. For example, in various embodiments ten parts may be effectively combined into one and only require two connectors to place into a bioprocess system. Embodiments of the invention may therefore provide a parts replacement ratio of >2:1, >3:1, >5:1, >10:1, etc.

As a result of the inventive design of the bioprocess system 1, the bioprocess system will be smaller and provide low hold up volume compared to prior art systems which is a great advantage for enhancing processing efficiency in the biopharmaceutical industry. Smaller holdup volume reduces the consumption and volume of buffers and solutions required for processing. Typically, a bioprocess comprises a larger number of process cycles, each cycle applying a sequence of different fluids to the separation or fluid treatment device in order to achieve the objective of the processing step. These fluids are provided via the different inlets of the bioprocessing system, alternatively, fluids and/or fluid properties may be altered already in an upstream processing step and/or system connected to one or multiple inlets of the bioprocessing system 1. The changeover of these fluids implies a waste of fluids and fluid volume as the efficiency of the changeover is directly related to the holdup volume of the bioprocessing systems and components. With the bioprocess system according to the invention, holdup volume is reduced drastically, and hereby separation quality and processing efficiency is increased and processing cost is reduced. Separation quality is improved by smaller hold up volume as larger hold up volume generally gives an adverse impact on the separation resolution that can be obtained with a chromatography setup. Hence, higher separation quality through smaller hold up volume increases the purity and yield in chromatography. Increased purity and yield lead in turn to increased efficiency of process, equipment and the manufacturing facility and operation. Reduced hold up volume also reduces time required for processing and pumping fluids as well as liquid volumes to be handled, hereby increasing processing efficiency, too. Hence, the system according to the invention provides increased separation and processing quality and/or increased processing efficiency, hereby providing eventually lower cost of drug substances and treatments provided to patients.

For example, various conventional bioprocess systems are known having a capacity from 5 L to 1,000 L, which have footprints ranging from 20 cm×21 cm to 1.6 m×1.5 m. Various embodiments of the present invention, however, can be used that enable the footprint of a bioprocess system to be reduced, for example, by at least 30% (e.g. 50% or even 70% or more). Hence, embodiments of the invention may have a footprint ranging from about 17 cm×17 cm (about 0.03 m²) to 1.3 m×1.3 m (about 1.7 m²) for bioprocess systems having a capacity from 5 L to 1,000 L (30% reduction); a footprint ranging from about 14 cm×14 cm (about 0.02 m²) to 1.1 m×1.1 m (about 1.2 m²) for bioprocess systems having a capacity from 5 L to 1,000 L (50% reduction); or a footprint ranging from about 11 cm×11 cm (about 0.01 m²) to 85 cm×85 cm (about 0.8 m²) for bioprocess systems having a capacity from 5 L to 1,000 L (70% reduction).

Embodiments of the present invention may thus provide reduced footprint bioprocess systems for bench systems and larger clean room-based systems. For example, it may be possible to reduce the footprint from 2.1 m×1.3 m for a system and 1.1 m×0.61 m for a pump cart to 1.1 m×1.1 m for the system, or even for both the system and pump cart together. Such smaller sized and smaller footprint systems are also easier to move, better fit through smaller doors etc., and are easier to incorporate into smaller clean room modules. Another advantage of the reduced holdup volume and the reduced footprint provided by the bioprocessing system according to the invention is that less clean room space is required compared to prior art bioprocessing systems. Hence, since clean room space is expensive, reduced drug/product manufacturing costs can be obtained.

Another advantage of the reduced footprint and holdup volume is that the bioprocess system according to the invention is improving the integration and connection of multiple unit operations and systems into a complete processing train and manufacturing setup. While the bioprocessing system as described so far is representing a single unit operation, with the example of a separation or fluid treatment system, this system is usually connected to other equipment upstream and downstream the unit operation. In a stand-alone unit operation and processing setup, typically associated with batch processing of a drug substance, inlet and outlet tanks may be connected for supplying and receiving fluid. The bioprocessing system according to the invention provides mentioned advantages of reduced holdup volume, footprint and processing space as already described. In another embodiment, the bioprocess system according to the invention may be part of a connected and/or continuous process setup where at least two unit operations and/or bioprocessing systems, usually set-up for different processing operations, are inter-connected to enhance overall process efficiency, reduce processing time, reduce footprint and/or provide a more or less continuous manufacturing setup and processing of product that eliminates intermediate fluid hold tanks otherwise required when running processes in a sequential and separated batch process setup, the latter corresponding to moving the product (drug substance) through a series of isolated and confined unit operations, which may even be physically run in different facilities and/or clean rooms. Thus, the bioprocessing system according to the invention improves process efficiency and reduces footprint in both batch, connected and (semi-)continuous processing setups.

In another embodiment of the invention, the bioprocessing system is compartmentalized into a fluid processing part and a control system part, where the control system part may be located outside the floorspace and processing footprint of the fluid processing system, outside the footprint of the clean room comprising the fluid processing system. In one example, the system control part, for example comprising electrical, pneumatic or electronics arrangements such as sensor transmitters, pump drive controllers, valve controllers, bus systems, controlling computers, processors (PLCs) etc. may be located separated from the fluid flow path, either in a separate cabinet inside the same clean room, thereby not obstructing the footprint of the fluid processing setup, or located outside the clean room in an adjacent typically non-classified room. A wall possibly provided in between the fluid processing part and the control system part may comprise cables and connectors for connection of the fluid processing part with the control system part. Parts of the system may also be located in the cloud, i.e. means for computing to achieve control and analysis of the processing system and process.

Another advantage of the bioprocessing system according to the invention providing low hold-up volume is that required cleaning procedures can be performed with higher efficiency and less fluid volume required. For cleaning, harsh and aggressive cleaning solutions are typically employed, such as caustic or oxidizing agents. These fluids are costly, both in production and disposal and extensive washing after cleaning operations is required. The reduced fluid volumes required by the system according to the invention will thereby reduce cost and environmental footprint in terms of requiring less energy and consumption of natural resources, such as water. It needs to be mentioned that water required for bioprocessing needs to be pre-treated by reverse osmosis or distillation and thereby high effort and energy consumption to obtain the WFI (water for injection) quality required for use in bioprocessing.

Beyond its low holdup volume, the bioprocessing system according to the invention also improves sanitization efficiency. This is achieved by the compact design with low hold-up volume, requiring lower volumes of sanitization fluids. Further, this is achieved by the system design with its vertical arrangement of the fluid path, hereby avoiding air trapped in the fluid path, which otherwise needs to be flushed out with extra fluid volume. Trapped air in a component may limit the access of the cleaning fluid to all internal surfaces of the fluid flow path and thereby cleaning efficiency. Air may be introduced at the inlets of the processing systems, for example when changing or re-connecting containers. However, air is also generated during the process by degassing of fluids leading to slow accumulation and generation of air pockets in the flow path, provided the flow path is not optimized and has sufficient physical inclination against the horizontal axis in the direction of flow such that air is conveyed toward the outlet of the system, hereby depleting the system from air.

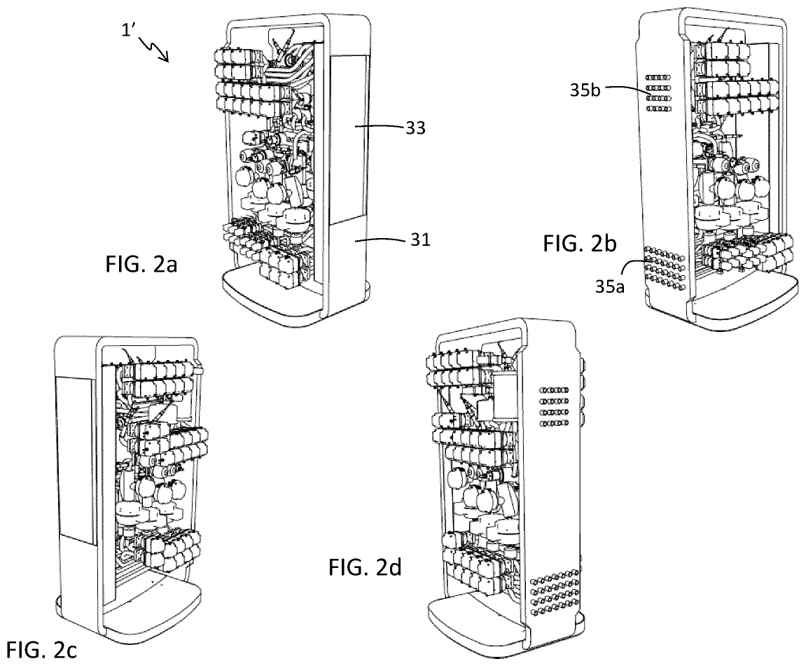

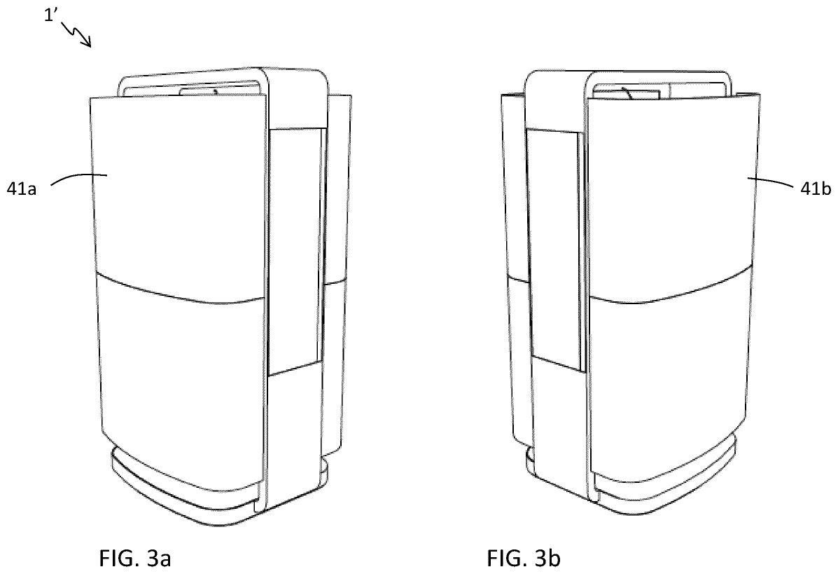

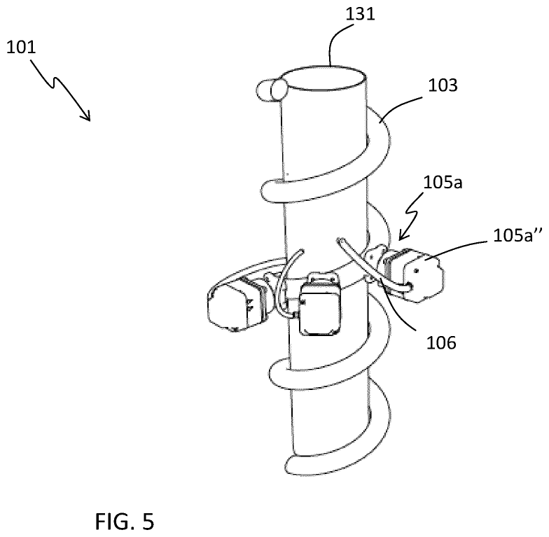

Furthermore according to some embodiments of the invention the flow path 3 is provided as a self-supporting flow path 3, at least to some degree. In some embodiments of the invention an additional support structure is also provided, for example as a supporting frame as will be described in relation to FIGS. 2 and 3, or as a support spine as will be described in relation to FIG. 5. However, in some embodiments of the invention the flow path 3 can be provided as a completely self-supporting structure. This can be achieved by printing the flow path 3 by a suitable thickness and by a suitable material. The thickness may vary for different parts of the flow path. Furthermore, in some embodiments of the invention the 3D printing of the flow path 3 can include printing some support structure outside the flow path 3 which will provide extra support.

All inlets and outlets 7, 9, 11 may be provided on the same side of the bioprocess system 1 as shown in FIG. 2b. However other arrangements of the inlets and outlets are of course possible.

In FIGS. 2a-2d a bioprocess system 1′ according to one embodiment of the invention is shown in different perspective views. The bioprocess system 1′ is almost identical with the bioprocess system 1 as described in relation to FIGS. 1a-1d. However, this bioprocess system 1′ further comprises a support structure 31 in the form of a partly surrounding frame. The flow path 3 can be connected to and supported by the support structure 31. Furthermore the support structure 31 can comprise a user interface 33 for control of the bioprocess system 1′. In this embodiment of the invention a user interface 33 is provided on one side of the support structure 31 and on the opposite side inlet and outlet fluid connections 35a, 35b to the inlets 7 and outlets 11 of the flow path 3 are provided.

In FIGS. 3a and 3b the bioprocess system 1′ as shown in FIGS. 2a-2d is provided with doors 41a, 41b. The doors can suitably be possible to open either by sliding them upwards or by attaching them by hinges on one side and swinging them outwards. The doors 41a, 4b may be transparent.

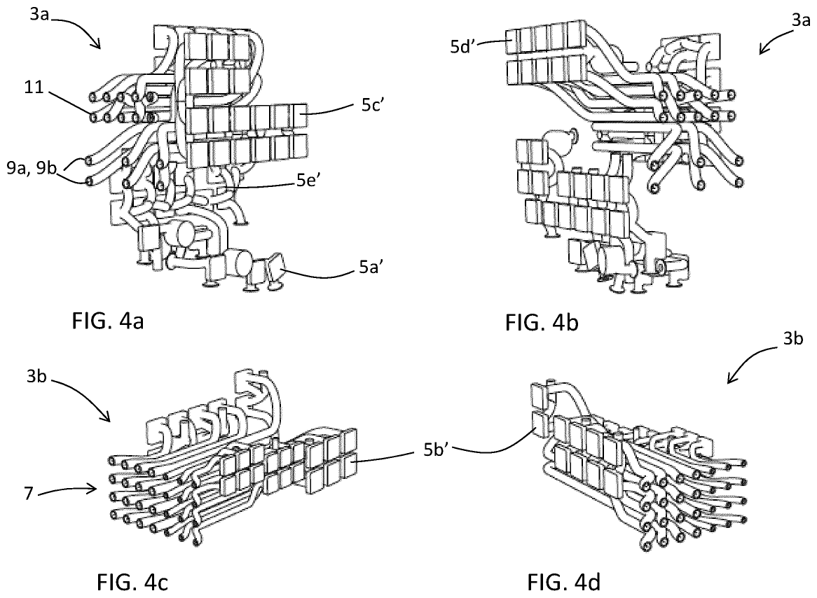

The flow path 3 as shown in FIGS. 1a-1d may be 3D printed in one or more sections. In FIGS. 4a-4d the fluid path 3 as shown in FIGS. 1a-1d is shown separated into two sections, an upper fluid path section 3a and a lower fluid path section 3b. Perspective views from a first and a second side are shown in FIGS. 4a-4d. The fluid path 3 can as well be printed in one single part or in more than two parts. The flow path 3 comprises as described above a plurality of inlets 7, at least one bioprocessing device inlet connection 9a, at least one bioprocessing device outlet connection 9b and a plurality of outlets 11. The flow path 3 comprises furthermore flow path parts 5a′, 5b′, 5c′, 5d′, 5e′ of active components provided in the bioprocess system 1 as discussed above and fluid paths between these described components. The whole flow path 3 or at least parts of the flow path 3 can as described above suitably be 3D printed. Some parts of the flow path 3 can possibly be produced in another way.

Separate sections of the flow path 3 can be provided according to the invention for being connected when assembling the bioprocess system 1. Hereby different types of sections can be combined in different ways for allowing different types and different sizes of systems to be provided. For example different number of inlets and outlets can be provided in the sections.

FIG. 5 shows a schematic view of a part of a bioprocess system 101 according to one embodiment of the invention. Here a support structure 131 is provided as a support spine vertically in the middle of the bioprocess system 101. The flow path 103 is provided around the support structure 131 and active parts 105a″ of active components 105 are mounted around the flow path 103 and are easily accessible from outside the bioprocess system 101. A cable 106 for electric or pneumatic control of the active component can be provided inside the support structure 131.

1. A bioprocess system comprising:
a flow path comprising:
a plurality of inlets;
at least one bioprocessing device inlet connection and at least one bioprocessing device outlet connection;
a plurality of outlets; and
flow path parts of at least some active components comprised in the bioprocess system: and
active parts of said active components,
wherein the flow path of the bioprocess system is positioned substantially centrally within the bioprocess system and in a substantially vertical orientation, whereby the active parts are positioned around the flow path being accessible from outside the bioprocess system and each in connection with a corresponding flow path part of an active component.

2. The bioprocess system according to claim 1, wherein the flow path is 3D printed in a number of sections or in one section.

3. The bioprocess system according to claim 1, wherein the flow path is at least to some degree self-supporting.

4. The bioprocess system according to claim 1, wherein the flow path is 3D printed with a thickness which is adapted such that the flow path is at least to some degree self-supporting, which thickness may vary for different parts of the flow path and/or wherein the flow path is 3D printed together with additional external support structures.

5. The bioprocess system according claim 1, wherein said active components comprise a number of valves and possibly also one or more pumps and one or more sensing components.

6. The bioprocess system according to claim 1, wherein said flow path is 3D printed from a corrosion resistant metal.

7. The bioprocess system according to claim 1, wherein the bioprocess system further comprises a support structure which comprises a central spine provided inside the flow path or a frame which is partly surrounding the flow path and which comprises user interface and tube connections.

8. The bioprocess system according to claim 1, having a footprint ranging from about 17 cm×17 cm to about 1.3 m×1.3 m for bioprocess systems having a capacity from 5 L to 1.000 L; a footprint ranging from about 14 cm×14 cm to about 1.1 m×1.1 m for bioprocess systems having a capacity from 5 L to 1.000 L; or a footprint ranging from about 11 cm×11 cm to about 85 cm×85 cm for bioprocess systems having a capacity from 5 L to 1.000 L.

9. The bioprocess system according to claim 1, having a footprint ranging from about 0.03 m² to about 1.7 m² for bioprocess systems having a capacity from 5 L to 1.000 L, a footprint ranging from about 0.02 m² to about 1.2 m² for bioprocess systems having a capacity from 5 L to 1.000 L. or a footprint ranging from about 0.01 m² to about 0.8 m² for bioprocess systems having a capacity from 5 L to 1,000 L.

10. The bioprocess system according to claim 1, wherein the flow path is at least partially 3D printed and provides a parts replacement ratio of >2:1, >3:1, >5:1 or >10:1.
